(19) United States
(12) Patent Application Publication
(10) Pub. No.: US 2008/0254046 A1
(43) Pub. Date: Oct. 16, 2008

(54) Method for the Delivery of Exogenous Antigens into the Mhc Class I Presentation Pathway of Cells

(76) Inventors: Christoph von Eichel-Streiber, Schweppenhausen (DE); Karina Gisch, Mainz (DE)

Correspondence Address: SALIWANCHICK, LLOYD & SALIWANCHIK;A PROFESSIONAL ASSOCIATION, P.O. BOX 142950, GAINSVILLE, FL, 32614-2950, US

(21) Appl. No.: 11/910,111

(22) PCT Filed: Apr. 7, 2006
(86) PCT No.: PCT/EP2006/003205

§ 371 Date: Jun. 15, 2008

Related U.S. Application Data
(60) Provisional application No. 60/670,052, filed on Apr. 11, 2005.

(30) Foreign Application Priority Data
Apr. 8, 2005 (DE) 10 2005 016 234.7

(51) Int. Cl. A61K 39/00; C12Q 1/02; A61P 37/00; C12Q 1/60

(52) U.S. Cl. 424/184.1; 435/29; 435/11

(57) ABSTRACT

The present invention relates to an in vitro method that allows for delivery of exogenous antigens into the MHC class I presentation pathway of antigen-presenting cells (APCs), comprising the following steps: (a) preparation of suitable APCs; (b) determination of suitable specific method parameters for APCs, comprising (1) bringing the cells in contact with a haemolysin such as listeriolysin (LLO) and a marker substance; (2) measuring of marker substance inflow into the cells; and (3) optionally, modifying said specific parameters; and (c) delivery of exogenous antigens into the APCs, by applying the specific parameters calculated in step (b) and bringing the cells in contact with a haemolysin and the antigen of interest.

The present invention relates to a two-stage in vitro method that allows for delivery of exogenous antigens into the MHC class I presentation pathway of antigen-presenting cells (APCs), comprising the following steps: (a) preparation of suitable APCs; (b) determination of suitable specific method parameters for APCs, comprising of (1) bringing the cells in contact with a hemolysin such as listeriolysin (LLO), and a marker substance; (2) measurement of marker substance inflow into the cells; and (3) optionally, appropriate modification of specific parameters; and (c) delivery of antigens into the MHC class I presentation pathway of antigen presenting cells, comprising of the direct transfer of the specific parameters determined in step (3) and comprising of bringing the cells in contact with hemolysin and the antigens of interest. The specific parameters can be selected in accordance with the following: the cell line or primary cell line into which the antigens are to be delivered; cell quality; cell concentration used; batch volume; culture medium and/or buffer used; the presence or absence of serum components during incubation; serum and/or cholesterol concentration; the antigen of interest; the laboratory material used; incubation temperature; cell line or primary cell line incubation time in the haemolysin; haemolysin type, purification method and concentration; haemolysin redox status during incubation.

The present invention uses haemolysin (and no other substance) to deliver antigens into the MHC CLASS I presentation-pathway of cells. Although the present invention is described in terms of a non-limiting example (LLO), other haemolysins such as those described in the literature (e.g. Provoda C. J., Lee K. D. 2000. Bacterial pore-forming hemolysins and their use in the cytosolic delivery of macromolecules. Adv. Drug Deliv. Rev. 41(2): 209-21. Welch R. A. 1991. Pore-forming cytolysins of gram-negative bacteria. Mol. Microbiol. 5(3): 521-8 oder Braun V., Focareta T. 1991. Pore-forming bacterial protein hemolysins (cytolysins). Crit. Rev. Microbiol. 18(2): 115-58) can also be used to deliver antigens into the MHC CLASS I presentation pathway. The terms haemolysin and LLO are used interchangeably in connection with the present invention.

For the purposes of the present invention all references as cited herein are incorporated by reference in their entireties.

The preferred embodiments of haemolysin are streptolysin O from Streptococcus pyogenes, S. equisimilis or S. canis, pneumolysin from S. pneumoniae, suilysin from S. suis, intermedilysin from S. intermedius, Listeriolysin from Listeria monocytogenes, ivanolysin from L. ivanovii, seeligerolysin from L. seeligeri, and other haemolysins from the thiol activated group of haemolysins (Alouf J. E. 2000. Cholesterol-binding cytolytic protein toxins. Int. J. Med. Microbiol. 290: 351-6).

### BACKGROUND OF THE INVENTION

Professional antigen-presenting cells, such as dendritic cells (DCs), are responsible for induction of a specific cellular immune response. Hence, stimulation of an immune response depends on the presence of antigens that are recognized as foreign by the host immune system.

Generally, antigen presenting cells take up extracellular antigen, which these cells then break down in their vesicular compartments. The antigens are then presented via MHC II molecules by a special type of T cells known as CD4⁺ T helper cells. On other hand, endogenous antigens are broken down with the aid of the proteasome and then presented to so called CD8⁺ T cells via MHC I molecules.

To induce a specific immune response against viruses, intracellular bacteria and tumor cells, it is necessary to realize targeted induction of a CD8⁺ cytotoxic T cell response. The discovery of tumor associated antigen has made it possible to use a host immune system to influence tumor growth. Whereas antigen-presenting cells can take up exogenous antigens very efficiently and can present the MHC class II restricted peptides thus generated, the currently known methods for targeted delivery of extracellular antigens for presentation with MHC class I molecules are time consuming, costly, and difficult to perform. MHC class I presentation of an antigen is a necessary precondition for induction of a CD8⁺ T cell response.

CD8⁺ cytotoxic T lymphocytes (CTLs) kill cells that that are infected with intracellular pathogens such as viruses, parasites or bacteria, and recognize specific peptides that are presented by MHC class I molecules. As a rule, MHC I molecules associate solely with peptides that arise in endogenously formed proteins.

On the other hand, professional antigen-presented cells (APCs) such as dendritic cells (DCs), macrophages and B cells have the capacity to process antigens from extracellular sources for presentation via MHC I molecules. This alternative MHC I molecule presentation pathway is also known as cross presentation and may play a significant role in the generation of CTL immunity (Rock K. L. 1996. A new foreign policy: MHC class I molecules monitor the outside world. Immunology Today 17: 129-137; Jondal M., Schirmbeck R. and Reimann J. 1996. MHC class I-restricted CTL responses to exogenous antigens. Immunity 5: 295-302; Yewdell J. W. 1999. Mechanisms of exogenous antigen presentation by MHC class I molecules in vitro and in vivo: Implications for generating CD8⁺ T cell responses to infectious agents, tumors, transplants, and vaccines. Adv. in Immunol. 73: 7-77; Ackerman A. L. and Cresswell P. 2004. Cellular mechanisms governing cross-presentation of exogenous antigens. Nat. Immunol. 5(7): 678-84).

APCs take up exogenous antigens via various mechanisms such as phagocytosis, macropinocytosis and receptor mediated endocytosis (Lanzavecchia A. 1996. Mechanisms of antigen uptake for presentation. Curr. Op. in Immunol. 8: 348-354). The uptake mechanism that is used influences antigen processing and presentation. High antigen concentrations are needed to activate CTLs via cross presentation of soluble antigens. The APCs take up these antigens via macropinocytosis or phagocytosis (Watts C. 1997. Capture and processing of exogenous antigens for presentation on MHC molecules. Ann. Rev. Immunol. 15: 821-850). MHC I presentation is strengthened by antigen aggregation, bead coupling or association with heat-shock proteins (Kovacsovics-Bankowski M. and Rock K. L. 1995. A phagosome to cytosol pathway for exogenous antigens presented on MHC class I molecules. Science 267: 243-246; Singh-Jasuja H. et al. 2000. Cross-presentation of glycoprotein 96-associated antigens on major histocompatibility complex class I molecules requires receptor-mediated endocytosis. J. Exp. Med. 191: 1965-1974; Castellino F. et al. 2000. Receptor-mediated uptake of antigen/heat shock protein complexes results in major histocompatibility class I antigen presentation via two distinct processing pathways. J. Exp. Med. 191: 1957-1964).

Antigen internalization via specific membrane receptors such as Fc or Mannose receptors also results in antigen cross presentation by APCs (Lanzavecchia A. 1996. Mechanisms of antigen uptake for presentation. Curr. Op. in Immunol. 8: 348-354; Regnault A. et al. 1999. Fcγ receptor-mediated induction of dendritic cell maturation and major histocompatibility complex class I-restricted antigen presentation after immune complex internalisation. J. Exp. Med. 189: 371-380). The processing and MHC-1 presentation of internalized antigens can be realized via the classic cytosolic proteasome and TAP dependent MHC I presentation pathway if antigens from endocytotic vesicles (endosomes or phagosomes) enter the cytosol. Alternatively, a cytosolic pathway can be used whereby pH adapted proteinases break down internalized antigens into peptides in the endocytotic vesicles. These peptides then bind to MHC-1 molecules and are transported to the cell surface as a complex. The MHC-1 molecules can be either regenerated molecules that arise in the plasma membrane, or newly synthesized complexes (Rock K. L. 1996. A new foreign policy: MHC class I molecules monitor the outside world. Immunology Today 17: 131; Castellino F. et al. 2000. Receptor-mediated uptake of antigen/heat shock protein complexes results in major histocompatibility class 1 antigen presentation via two distinct processing pathways. J. Exp. Med. 191: 1957-1964; Rodriguez A., Regnault A., Kleijmeer M, Ricciardi-Castagnoli P. and Amigorena S. 1999. Selective transport of internalized antigens to the cytosol for MHC class I presentation in dendritic cells. Nature Cell Biol. 1: 362-368, Gromme M. et al. 1999. Recycling MHC class I molecules and endosomal peptide loading. Proc. Nat. Acad. Sci. (USA) 96: 10326-10331).

DCs are the only APCs that stimulate naive CD8⁺ T lymphocytes and a CTL response (Banchereau J. and Steinman R. M. 1998. Dendritic cells and the control of immunity. Nature 392: 245-252). Immature DCs in peripheral tissues take up exogenous antigens from various sources including microbes, infected cells, cell debris, proteins and immune complexes. Antigen loaded DCs migrate toward the secondary lymphoid organs, process antigens for purposes of antigen presentation, and during this migration process acquire the ability to attract and activate dormant CD8⁺ T cells.

Hence, MHC I presentation of exogenous antigens by DCs is the precondition for stimulation of a CTL response against tumors, intracellular bacteria, or viral antigens that do not infect APCs. The role of cross presentation in a murine polio virus infection model has been investigated in vivo. In order to induce CTL immunity against the aforementioned virus (which is not replicated in APCs), it is necessary to induce MHC I presentation of extracellular viral antigens (Yewdell J. W. 1999. Mechanisms of exogenous antigen presentation by MHC class I molecules in vitro and in vivo: Implications for generating CD8⁺ T cell responses to infectious agents, tumors, transplants, and vaccines. Adv. in Immunol. 73: 7-77; Albert M. L., Sauter B., and Bhardwaj N. 1998. Dendritic cells acquire antigen from apoptotic cells and induce class I-restricted CTLs. Nature 392: 86-89; Sigal L. J., Crotty S., Andino R. and Rock. K. L. 1999. Cytotoxic T cell immunity to virus-infected non-haematopoietic cells requires presentation of exogenous antigen. Nature 398: 77-80; Yewdell J. W., Bennink J. R. and Hosaka Y. 1988. Cells process exogenous proteins for recognition by cytotoxic T lymphocytes. Science 239: 637-640; Reimann J. and Schirmbeck R. 1999. Alternative pathways for processing exogenous and endogenous antigens that can generate peptides for MHC class I-restricted presentation. Immunol. Rev. 172: 131-152).

Various humoral and cellular immunity activation mechanisms for tumor therapy are currently under investigation. Some elements of cellular immunity have the capacity to recognize and destroy specific tumor cells. Isolation of CTLs from tumor derived cell populations or from peripheral blood suggests that such cells play a key role in natural immunity to cancer (Cheever et al 1993. Annals N.Y. Acad. Sci. 690: 101-112). There are already numerous examples of both murine and human CTLs that recognize specific tumor cells and exhibit therapeutic activity following adoptive transfer, and in some cases induce full remission. However, the progressive growth of most cancers shows that in spite of the inherent ability of CD8⁺ T cells to destroy tumor cells, many of these tumors escape recognition by CTLs in vivo. It has proven difficult thus far to induce activation in vivo of a sufficient number of CD8⁺ T cells (Burns D. M. and Crawford D. H. 2004. Epstein-Barr virus-specific cytotoxic T-lymphocytes for adoptive immunotherapy of post-transplant lymphoproliferative disease Blood Rev. 18(3): 193-209; Kawai K., Saijo K., Oikawa T., Morishita Y., Noguchi M., Ohno T. and Akaza H. 2003. Clinical course and immune response of a renal cell carcinoma patient to adoptive transfer of autologous cytotoxic T lymphocytes. Clin. Exp. Immunol. 134(2): 264-9; Bathe O. F., Dalyot-Herman N. and Malek T. R. 2003. Therapeutic limitations in tumor-specific CD8⁺ memory T cell engraftment. BMC Cancer 3(1): 21).

Most protein delivery methods into the cytosol that aim to preserve cell viability are time consuming and difficult to perform, and none have been completely successful thus far. No extra-laboratory method for use in a clinical setting is known to date. Although delivery can be realized via microinjection, this is a technically demanding method that can only be performed on a limited number of cells (Schneider G. B., Gilmore A. P., Lohse D. L., Romer L. H., Burridge K. 1998. Microinjection of protein tyrosine phosphatases into fibroblasts disrupts focal adhesions and stress fibres. Cell Adhes. Commun. 5(3): 207-219; Gorbsky G. J., Chen R.-H. and Murray A. W. 1998. Microinjection of Antibody to Mad2 Protein into Mammalian Cells in Mitosis Induces Premature Anaphase J. Cell Biol. 141: 1193-1205). Electroinjection appears to be a promising technique, but it unsuitable for adherent cells (Wilson A. K., Horwitz J. and De Lanerolle P. 1991. Evaluation of the electroinjection method for introducing proteins into living cells. Am. J. Physiol. 260: C355-63).

Additional methods for the delivery of extracellular macromolecules into the cytosol and MHC I presentation pathway have been described. All of these methods require considerable methodological expertise and none is widely used (Bungener L., Huckriede A., Wilschut J. and Daemen T. 2002. Delivery of Protein Antigens to the Immune System by Fusion-active Virosomes: A Comparison with Liposomes and ISCOMS. Bioscience Reports 22: 323-338). The “trojan” peptide penetration system can only be realized with oligopeptides (Derossi D., Chassaing G. and Prochiantz A. 1998. Trojan peptides: the penetration system for intracellular delivery. Trends Cell Biol. 8(2): 84-7). The viral hemagglutinin mediated fusion technique works well but is difficult to perform (Doxsey S. J., Sambrook J., Helenius A. and White J. 1985. An efficient method for introducing macromolecules into living cells. J. Cell Biol. 101(1): 19-27).

Virus like particles (VLPs) constitute another option for in vivo or in vitro delivery of antigen epitopes to the MHC I presentation pathway. Here, the antigen is embedded in the VLPs. In the case of DCs, the VLPs are take up, whereupon the antigens embedded in the VLPs are transported from the endosome to the cytosol and are presented with MHC I molecules, thus enabling the DCs to activate CD8+ T cells.

In the case of DCs, following VLP internalization, the antigens obtained from the endosome enter the cytosol and are presented with MHC class I molecules, thus giving the DCs have the capacity to activate CD8+ T cells. One drawback of this method is the time consuming VLP generation and purification process (Moron V. G., Rueda P., Sedlik C. and Leclerc C. 2003. In vivo Dendritic Cells can cross-present Virus-like Particles using an Endosome-to-Cytosol Pathway. J. Immunol. 171: 2242-2250; Storni T., Lechner F., Erdmann I., Bächi T., Jegerlehner A., Dumrese T., Kündig T. M., Ruedl C. and Bachmann M. F. 2002. Critical Role for Activation of Antigen-presenting cells in Priming of Cytotoxic T Cell Response After Vaccination with Virus-Like Particles. J. Immunol. 168: 2880-2886).

WO 02/072140 describes an immunogenic compound that has the capacity to induce in vitro or in vivo CTL response to a viral disease via MHC I presentation of exogenous antigens without the necessity for viral replication. The compound contains a virus, whose infectious properties are deactivated or attenuated. The virus has the capacity to fuse with the cells.

Like VLPs, PLGA-(poly(lactic-co-glycolic acid)) particles have the capacity to infiltrate encapsulated peptides or proteins in the MHC I presentation pathway. As with VLPs, the particle generation process is time consuming (Waeckerle-Men Y. and Groettrup M. 2005. PLGA microspheres for improved antigen delivery to dendritic cells as cellular vaccines. Adv. Drug Deliv. Rev. 57(3): 475-82; Zheng C. H., Gao J. Q., Zhang Y. P. and Liang W. Q. 2004. A protein delivery system: biodegradable alginate-chitosan-poly(lactic-co-glycolic acid) composite microspheres. Biochem. Biophys. Res. Commun. 323(4): 1321-7).

Cationic lipids are frequently used to deliver proteins into the cell cytosol, where the lipids and soluble proteins form a complex that binds to the negatively charged cell surfaces.

This method is advantageous in that only small amounts of protein are needed. Insufficient interaction with cationic lipids sometimes causes problems with positively charged molecules (Simberg D., Weisman S., Talmon Y. and Barenholz Y. 2004. DOTAP (and other cationic lipids): chemistry, biophysics, and transfection. Crit. Rev. Ther. Drug Carrier Syst. 21(4): 257-317; Zelphati O., Wang Y., Kitada S., Reed J. C., Felgner P. L. and Corbeil J. 2001. Intracellular delivery of proteins with a new lipid-mediated delivery system. J. Biol. Chem. 276: 35103-110).

U.S. Pat. No. 5,643,599 describes a method that allows extracellular antigens to be delivered into the target cell cytosol using liposomes. In addition to the antigens delivered, the liposomes contain a substance (e.g. a haemolysin) that can permeabilize the phagosomal membrane after the liposomes have been take up via phagocytosis and their content has been released into the phagosome, thus enabling extracellular antigens to access the cytosol. This method allows exogenous antigens to infiltrate cells that have the capacity to internalize liposomes.

In contrast to the foregoing, the current invention involves the use of soluble haemolysin. Moreover, the present method also allows for the delivery of antigens into the MHC class I presentation pathway of non-phagocytizing cells.

WO 01/87325 describes a method that allows for an increase in MHC class I presentation of soluble tumor or tissue antigens using human dendritic cells (DCs). Before being administered to tumor patients, DCs from human donors are incubated with soluble antigens in combination with bacille Calmette Guérin (BCG). The presence of BCG during DC incubation with soluble exogenous antigens leads to a higher level of processing and presentation of antigen epitopes with MHC class I molecules, relative to that obtained when antigens are incubated in the absence of BCG. Following in vivo administration of DCs that are pretreated with both antigens and adjuvant BCG, a larger number of antigen-specific CTLs are activated than following administration of DCs that are pre-incubated with antigens only.

Various attempts have been made to realize oral or parenteral immunization in murine models using recombinant Listeria or Salmonella that express tumor associated antigens. In these experiments, it was observed that the animals were either protected against tumors or that the tumors regressed, both of which effects are attributable to the induction of tumor antigen-specific CTLs (Cochlovius B., Stassar M. J., Schreurs M. W., Benner A. and Adema G. J. 2002. Oral DNA vaccination: antigen uptake and presentation by dendritic cells elicits protective immunity. Immunol. Lett. 80(2): 89-96; Weth R., Christ O., Stevanovic S, and Zoller M. 2001. Gene delivery by attenuated Salmonella typhimurium: comparing the efficacy of helper versus cytotoxic T cell priming in tumor vaccination. Cancer Gene Ther. 8(8): 599-611; Paterson Y. and Johnson R. S. 2004. Progress towards the use of Listeria monocytogenes as a live bacterial vaccine vector for the delivery of HIV antigens. Expert Rev. Vaccines 3(4Supl.): 119-134; Sewell D. A., Douven D., Pan Z. K. Rodriguez A. and Paterson Y. 2004. Regression of HPV-positive tumors treated with a new Listeria monocytogenes vaccine. Arch. Otolaryngol. Head Neck Surg. 130(1): 92-7; Lin C. W., Lee J. Y., Tsao Y. P., Shen C. P., Lai H. C. and Chen S. L. 2002. Oral vaccination with recombinant Listeria monocytogenes expressing human papillomavirus type 16 E7 can cause tumor growth in mice to regress. Int. J. Cancer 102(6): 629-637).

U.S. Pat. No. 6,565,852 describes a method that allows for induction of an immune response against tumor associated antigens by administering a recombinant form of Listeria monocytogenes that express a tumor associated antigen or fragment thereof. The tumor associated antigen can be expressed by recombinant listeria as a standalone protein or as a listeriolysin fusion protein. U.S. Pat. No. 5,830,702 describes the use of living recombinant Listeria monocytogenes for the induction of a cytotoxic T cell response using an attenuated stem of Listeria spp. that expresses a specific foreign antigen. This same patent also describes methods allowing for the induction of protective immunity by administering an efficacious quantity of the Listeria vaccine.

U.S. Pat. No. 6,051,237 describes a tumor specific immunotherapy using a living recombinant bacterial vaccine vector in the form of recombinant Listeria monocytogenes that express tumor specific antigens.

The therapeutic use of genetically modified living vectors with human pathogenic potential is controversial. The method is particularly risky for patients whose immune functions have been suppressed by infections such as HIV or tumor disease (Redfield R. R., Wright D. C., James W. D., Jones T. S., Brown C. and Burke D. S. 1987. Disseminated vaccinia in a military recruit with human immunodeficiency virus (HIV) disease. N. Engl. J. Med. 316: 673-676; Kavanaugh D. Y. and Carbone D. P. 1996. Immunologic dysfunction in cancer. Hematol. Oncol. Clin. North Am. 10: 927-951).

Radford et al (Radford K. J., Higgins D. E., Pasquini S., Cheadle E. J., Carta L., Jackson A. M., Lemoine N. R., Vassaux G. A recombinant E. coli vaccine to promote MHC class I-dependent antigen presentation: application to cancer immunotherapy. Gene Ther. 2002: 9(21): 1455-63; Radford K. J., Jackson A. M., Wang J. H., Vassaux G., Lemoine N. R. Recombinant E. coli efficiently delivers antigen and maturation signals to human dendritic cells: presentation of MART1 to CD8⁺ T cells. Int. J. Cancer. 2003: 105(6): 811-9) describe the induction and activation of antigen-specific CTLs in both human (2002) and murine (2003) model systems by infecting DCs with recombinant E. coli that express a model antigen and LLO. In the murine experiments, DCs were first pulsed with E. coli expressing LLO and OVA. These pretreated DCs were then injected into murine models, which resulted in the activation of OVA specific CTLs that had the capacity to inhibit growth of an OVA expressing tumor. Building on these results, E. coli that express the melanoma antigen MART-1 were used in conjunction with LLO in human DCs. Following incubation with paraformaldehyde fixed MART-1/LLO expressing E. coli, human DCs exhibited phenotypical and functional maturity. Following the pre-treatment described above, the DCs had the capacity to activate MART-1 specific CTLs, which was attributable to presentation of the MHC class I restricted peptides MART-1 27-35. Radford et al discuss the potential for this system to become a new strategy for human tumor immunotherapy. The system comprises recombinant antigen-expressing E. coli and entails cloning of the antigen that is to be presented in the MHC class I pathway. This method for the delivery of exogenous antigens into the MHC class I presentation pathway can only be used with cells can internalize living or fixed E. coli. In contrast to the foregoing, the current method involves treatment using soluble LLO and can also be employed with non-phagocytizing cells to deliver antigens into the MHC I presentation pathway.

None of the methods described above provide a simple and replicable method for the rapidly replicable and efficacious delivery of exogenous antigens into the MHC class I processing pathway of various cells.

Accordingly, it is an object of the present invention to provide a method allowing for the effective and replicable delivery of exogenous antigens into the MHC class I presentation pathway for the induction or activation of antigen-specific CD8⁺ T cells.

The first embodiment of the present invention fulfils this task by means of a simple and universally applicable two-stage in vitro method allowing for the delivery of antigens by reversible permeabilization of potential antigen-presenting cells via any cholesterol-binding haemolysin such as Listeriolysin (LLO; Palmer M. 2001. The family of thiol-activated, cholesterol-binding cytolysins. Toxicon 39(11): 1681-9; Alouf J. E. 2000. Cholesterol-binding cytolytic protein toxins. Int. J. Med. Microbiol. 290(4-5): 351-6). The terms hemolysin and LLO are used interchangeably in connection with the present invention, which relates to a two-stage in vitro method for the delivery of exogenous antigens into the MHC class I presentation pathway of antigen-presenting cells (APCs), comprising of the following steps: (a) preparation of suitable antigen-presenting cells; (b) determination of suitable specific method parameters for antigen-presenting cells, comprising of (1) bringing the cells in contact with a haemolysin such as Listeriolysin (LLO) and a marker substance; (2) measurement of marker substance inflow into the cells during and/or after treatment with LLO-; and (3) optionally, appropriate modification of the aforementioned specific parameters; c) delivery of antigens into the MHC class I presentation pathway of antigen presenting cells, comprising of the direct transfer of the specific parameters determined in step (3) and comprising of bringing the cells in contact with LLO and the antigens of interest (contact with the antigens occurs during or following incubation with LLO).

Accordingly, the present invention generally comprises the following elements whose purpose is to adapt the efficacy of LLO to defined conditions (keeping all parameters except LLO concentration and incubation time constant): testing the conditions (concentration and incubation time) under which LLO is used with the aid of a marker substance such as PI that is readily amenable to direct observation. In this testing method, the marker substance is added during or after incubation with LLO, and marker substance inflow into the cells is measured. If excessive marker substance flows into the cells, LLO concentration or incubation time is shortened. If no marker substance flows into the cells, LLO concentration or incubation time is increased. These tests take 15-45 minutes each. Once the relevant parameters have been identified, they are applied to antigen delivery. In a preferred embodiment of the invention, the method described above first entails measurement of propidium iodide (PI) inflow under defined conditions in potential antigen-presenting cells during or after Listeriolysin activity. As a rule, optimal LLO concentration and incubation time occur upon incipient PI inflow (shortly before the commencement of PI inflow, upon commencement of PI inflow, upon demonstrable PI inflow, depending on cell sensitivity). The parameters thus determined are then applied to LLO treatment for purposes of antigen delivery. This process renders the method replicable and for the first time enables rapid and reliable application, particularly in clinical settings. Thus, transferring the exact conditions of the preliminary tests using PI to the incubation of peripheral blood mononuclear cells (PBMCs) with LLO and the antigens of interest leads to a greater than 100% increase in the concentration of demonstrably antigen-specific CD8⁺ cells (see example 5).

Streptolysin O (SLO), which is a prototype of the cholesterol-binding family of bacterial exotoxins, is a pore forming protein that forms pores in the plasma membrane of macrophages. The three dimensional structure of perfringolysin, which is also a member of the aforementioned family, has been described, and the molecular mechanisms leading to pore formation via SLO are partially understood (Sekiya K., Danbara H., Yase K. and Futaesaku Y. 1996. Electron microscopic evaluation of a two-step theory of pore formation by streptolysin O. J. Bacteriol. 178(23): 6998-7002; Heuck A. P., Tweten R. K. and Johnson A. E. 2003. Assembly and topography of the prepore complex in cholesterol-dependent cytolysins. J. Biol. Chem. 278(33): 31218-225). After binding to the membrane, hemolysin monomers diffuse laterally in the bilayer and oligomerise, forming homotype aggregates that display very large transmembrane pores with diameters ranging up to 35 mm. SLO mediated pore formation is generally lethal for the target cells when conventional protocols are used, and thus only a limited number of post-permeabilization cell biology tests can be realized before cells die. However, SLO was used in several experiments to test cellular processes for short periods.

Walev et al (Walev I., Bhakdi S. C., Hofmann F., Djonder N., Valeva A., Aktories K., Bhakdi S. 2001. Delivery of proteins into living cells by reversible membrane permeabilization with streptolysin-O. Proc. Natl. Acad. Sci. USA 98(6): 3185-90) describe the pore forming toxin streptolysin O (SLO), which can be used for reversible permeabilization of adherent and non-adherent cells, thus allowing molecules with a mass of up to 100 kDa to enter the cytosol. The authors estimated (using FITC marked albumin) that an uptake rate of 10⁵-10⁶ molecules per cell is achieved. They also found that Ca(2+) calmodulin and intact microtubules are needed for the repair of toxin lesions, and that the active domains of large clostridial toxins could be delivered into three different cell lines via the SLO pores. The authors discuss in general terms the broad applicability of their method, but do not mention antigen delivery accompanied by subsequent MHC class I presentation.

Darji et al describe a method to use soluble proteins for the in vivo and in vitro stimulation of CD8⁺ T cells by utilizing the pore formation activity of LLO (Darji A., Chakraborty T., Wehland J. and Weiss S. 1995. Listeriolysin generates a route for the presentation of exogenous antigens by major histocompatibility complex class I. Eur. J. Immunol. 25(10): 2967-71) Immunization using active soluble hemolytic listeriolysin induces both CD8⁺ and CD4⁺ LLO-specific T cells, whereas heat activated LLO only induces CD4⁺ LLO specific T cells. Thus, active haemolytic LLO induces self-delivery into the MHC class I presentation pathway. In addition, model antigens such as ovalbumin, admixed with Listeriolysin, are also delivered into the MHC class I presentation pathway in vitro and in vivo.

In a similar vein, Darji et al (Darji A., Chakraborty T., Wehland J. and Weiss S. 1997. TAP-dependent major histocompatibility complex class I presentation of soluble proteins using Listeriolysin. Eur. J. Immunol. 27(6): 1353-9) describe the immunization of mice using Listeriolysin and antigens such as soluble ovalbumin, influenza virus nucleoprotein or beta galactosidase from Escherichia coli. In vivo, this results in the induction of a cytotoxic CD8⁺ T cell response against defined MHC class I-restricted peptide epitopes of these proteins. In vitro, the treatment of established cell lines using LLO and the antigens mentioned above leads to antigen delivery into the MHC class I presentation pathway. The presentation of MHC class I-restricted peptides from the antigens was detected by proliferating antigen-specific T cell clones. LLO mediated delivery in the MHC class I presentation pathway was dependent on the presence of a functional TAP transporter and could be inhibited using brefeldin A. This suggests that exogenous LLO allows antigens to access the cytosol as well as the classic MHC class I presentation pathway. Darji et al report that target cell treatment using Listeriolysin under the experimental conditions they selected had no impact on cell viability. The pores generated by Listeriolysin treatment were repaired within 60 minutes. The authors' method for delivery of soluble proteins into the MHC class I presentation pathway provides a system that allows for investigations of the cytotoxic response against intracelluar pathogens, and would also allow for screening of potential antigens in vaccine formulations.

Darji et al describe in vitro LLO based antigen delivery solely into EL-4 and P815 cells (murine cell lines), as well as in an animal model. These authors used primary murine spleen cells to investigate the effect of LLO on MHC CLASS II presentation, and did not use LLO for sensitive primary cells such as human PBMCs.

An attempt by the inventor to replicate Darji et al's experiment showed that their technique is extremely labour intensive and time consuming, requires substantial quantities of material, and fails in all but a few isolated instances. It took the present inventor over two years (with Dr. Darji's technical assistance) to reproduce partially Darji's published results. Against the backdrop of the extensive experiments realized in connection with the present invention, it was determined that the inefficacy of Darji et al's method is attributable to the following factors:
The quantity of LLO that is needed to induce pore formation in APCs using LLO and deliver antigens into eukaryotic cells is so large that it has an almost toxic effect.
Using an unduly large amount of LLO kills the treated cells, which are then of course unable to process the antigen that is delivered into them.
If too little LLO is used, either no pores form or the pores that do form lack the properties needed to deliver a sufficient quantity of antigens into the cytosol for presentation with MHC I molecules.

In using Darji et al's application method for LLO the present authors found that a substantial number of time consuming experiments are required to reproducibly deliver proteins into the MHC class I pathway. Consequently, Darji et al's method is unsuitable for research or for routine laboratory applications. Darji et al provide no universally applicable methodological guideline that describes how LLO mediated pore formation can be induced in cell membranes; nor do they provide a method for direct observation of pore formation. Hence Darji et al's antigen delivery method necessitates time consuming and costly experiments for each new batch and for even minute changes in experimental conditions.

The inventor's investigations also show that the following parameters have an impact on experimental outcomes:
the cell lines or primary cells in which the antigen is to be delivered
the quality of the cells used
the cell concentration used
batch volume
the culture medium and/or buffer used
medium or buffer pH value
the presence or absence of serum components
serum and/or cholesterol concentration
the properties and purity of the antigen under investigation
the laboratory material used
reaction temperature
LLO type
LLO purification method
LLO redox status

Hence, in order for the method of haemolysin mediated delivery of soluble proteins to the MHC I pathway of cell to be suitable for laboratory research and clinical routines, it must be optimized in such a way that LLO mediated delivery of proteins can be readily adapted to altered conditions.

The goal of the present invention is to provide a method that allows for rapid and simple analysis of the numerous factors that affect LLO efficacy so as to allow for simple and rapid adaptation to laboratory conditions. Another goal of the present invention is to provide a resilient and flexible method that can be used for clinical routines with a broad range of specimens and personnel.

In a preferred embodiment of the method according to the present invention, effective LLO concentration and incubation time are determined by measuring PI inflow during or after LLO incubation. This may be done by treating the cells with a defined LLO concentration that appears to be optimal for the defined experimental conditions. Iterative measurements are realized for a predefined time period ranging from 10-45 minutes until PI inflow into the target cells is detectable.

“Defined experimental conditions” means the conditions selected by the experimentator such as the desired cell concentration, incubation temperature, medium and so on. As a rule, the optimal LLO concentration and incubation time for antigen delivery into the MHC class I presentation pathway are that associated with the incipient PI inflow under the defined conditions. According to the present invention, this method provides the advantage that the aforementioned multifactorial impact of the effective LLO concentration becomes negligible or is eliminated entirely by transferring the previously determined reaction parameters to the exogenous antigen delivery method, while all other parameters remain constant. This method renders the tests replicable. An additional advantage provided by the method is that it allows for rapid transfer of the LLO application to another laboratory where other conditions may exist or be desired.

PI flow is used as an example and a preferred embodiment within the framework of the present invention for purposes of identifying the range of action, within which LLO can be utilised for antigen delivery into the cytosol followed by processing and presentation with MHC class I. In the application example described here, the number of activated antigen-specific CD8⁺ T cells increases coterminously with the LLO-mediated inflow of PI into the cells in the presentation experiment, providing that the toxicity threshold has not been reached (example 4).

Additional advantages provided by the method according to the present invention are as follows: adherent as well as suspension cells can be treated successfully using LLO and antigens; fresh or cryopreserved cells can be used; and the treatment can be realized with or without serum in the various media or buffers. In contrast to the method described by Darji et al, the method according to the present invention greatly reduces (to a matter of weeks) the amount of time needed to initially establish LLO mediated delivery of exogenous antigens to the MHC class I presentation pathway. As marker inflow is amenable to synchronous (live) observation, the adaptation to the laboratory's conditions (e.g. adaptation to the cell line used, adaptation to the laboratory materials, establishment of a suitable temperature and so on) is easily achieved. Thus the present invention allows for the rapid determination of the optimally efficacious LLO concentration and incubation time in rapid preliminary tests. In order to deliver soluble antigens to the MHC class I presentation pathway the parameters determined in the preliminary test are simply transferred to the incubation of cells with antigen. The parameters of the preliminary tests can be altered in any way desired. If parameters such as cell concentration or LLO batch are modified, the effective LLO concentration and incubation time can be determined expeditiously and sensitively by measuring PI inflow.

The method according to the present invention also allows for the following:

1. Exogenous antigens can be delivered into primary cells, mouse cells and human cells (The description of the in vitro method by Darji et al refers only to established cell lines and makes no mention of the use of primary cells.).
2. Exogenous antigens can be delivered into non-separated human peripheral blood mononuclear cells (PBMCs) (see examples 4-6). This is a key precondition for clinical applications of LLO, such as the determination of patient CTL responsiveness or the expansion of antigen-specific CTLs. In clinical test situations, such methods must be amenable to expeditious and simple realization. However owing to the method's lack of replicability and the time consuming tests involved, Darji et al's method cannot be used for routine applications or in a clinical trial setting.
3. Exogenous antigens can be delivered into human monocyte lines (THP-1) or primary human monocytes.

Accordingly, a key factor in this regard is the development of a method that allows for the expeditious and sensitive cell type-specific determination of the effective LLO concentration and incubation time by measuring a marker such as propidium iodide (PI) inflow during or after haemolysin activity under defined conditions.

When the defined conditions (parameters) under which an effective marker inflow is demonstrable are transferred to the antigen incubation method, no allowance need be made for the aforementioned operant parameters. In terms of the present invention, this means a “direct transfer of the specific parameters that have been determined.”

Another preferred embodiment of the present invention is an in vitro method for antigen delivery into the MHC class I presentation pathway of any type of cell with MHC class I presentation capacities, particularly into animal cells such as primary human and murine cells; cell lines; non-separated or separated cells such as peripheral blood mononuclear cells (PBMCs), monocytes or lymphocytes; immature monocyte derived DCs; the THP-2 cell line, mouse bone marrow in suspension; adherent mouse bone marrow macrophages; the EL-4 cell line; and the NIH3T3 cell line.

Another preferred embodiment of the present invention is an in vitro method for the delivery of antigens into the MHC class I presentation pathway, whereby the antigen is selected from among those proteins or the components thereof whose antigen epitope is to be presented with MHC I molecules. Examples of such antigens can be found in the Cancer Immunity Peptide Database and the MHCBN Database (available at http://www.imtech.res.in/raghava/mhcbn/; or http://bioinformatics.uams.edu/mirror/mhcbn), and include tumor associated antigens, viral antigens, intracellular bacteria antigens, as well as model antigens such as ovalbumin, CMVpp65 and components thereof. “Components” within the meaning of the present invention comprise all peptides, oligopeptides, proteins and fusion proteins that are large enough to be delivered into MHC class I presentation pathway cells. The size of such peptides ranges up to 540 kDa, and is preferably between 5 kDa and 200 kDa, and even more preferably between 10 kDa and 50 kDa. Lund et al (2002. Web-based tools for vaccine design in: Korber B., Brander C., Haynes B., Koup R., Kuiken C., Moore J., Walker B. and Watkins D., eds., HIV Molecular Immunology 45-51. Theoretical Biology and Biophysics Group, Los Alamos National Laboratory, Los Alamos N. Mex.) provide an overview of the various peptide epitope databases.

Another embodiment of the present invention is an in vitro method for the delivery of antigens into the MHC class I presentation pathway, whereby an additional specific parameter is the size, load and/or amino acid composition of the antigen. Antigens that are toxic for the target cells and hence are not processed or presented can be used in an inactivated form (following mutagenesis or chemically inactivated).

Another preferred embodiment of the present invention is an in vitro method for the delivery of antigens into the MHC class I presentation pathway, whereby the LLO is selected from culture supernatant, purified LLO, or recombinantly produced LLO. Such methods are known to a person skilled in the art and are found in the literature, Giammarini et al (J. Biotechnol. 2004: 109(1-2): 13-20), Walton et al. (Protein Expr. Purif. 1999: 15(2): 243-5), Traub und Bauer (Zentralbl. Bakteriol. 1995: 283(1): 29-42) und Darji et al. (J. Biotechnol. 1995: 43(3): 205-12) being one example.

Another preferred embodiment of the present invention is an in vitro method for the delivery of antigens into the MHC class I presentation pathway, whereby a dye and/or fluorescent compound such as propidium iodide (PI, in a concentration ranging from 1-10 μg/ml) or comparable material is used as a marker. Other suitable markers include 7-AAD, eGFP, and fluorescence marked proteins such as BSA-FITC, BSA-PE, and OVA-FITC.

Another preferred embodiment of the present invention is an in vitro method for the delivery of antigens into the MHC class I presentation pathway, whereby marker inflow is measured fluorescently. Additional suitable measurements include measurement of the inflow of ions such as calcium ions; measurement of changes in membrane polarization; release of intracellular components such as lactate-dehydrogenase (LDH); comparison of the cell proliferation rate following haemolysin (e.g. LLO) and the cell proliferation rate of a control tested without haemolysin; proliferation measurement via integration of tritium marked thymidine in DNA by cleavage or conversion of a substrate such as MTT, XTT or WST-1; or other methods that are known to a person skilled in the art.

Another preferred embodiment of the present invention is an in vitro method for the delivery of antigens into the MHC class I presentation pathway, whereby LLO incubation time ranges from 1 minute to 16 hours, and mainly ranges from 1-45 minutes, and particularly from 1-20 minutes.

Another preferred embodiment of the present invention is an in vitro method for the delivery of antigens into the MHC class I presentation pathway, whereby LLO concentration ranges from 1 to 5000 ng/ml, mainly ranges from 1 to 500 ng/ml, and preferably ranges from 1 to 250 ng/ml.

Another preferred embodiment of the present invention is an in vitro method for the delivery of antigens into the MHC class I presentation pathway, whereby cell concentrations ranging from 10⁵/ml to 10⁹/ml are used, and the preferred concentrations range from 5×10⁵ to 5×10⁶/ml.

Another preferred embodiment of the present invention is an in vitro method for the delivery of antigens into the MHC class I presentation pathway, whereby the composition and sources of the culture medium and/or buffer used vary, e.g. PBS, DMEM and RPMI. Additional buffers and media that can be used are known to a person skilled in the art.

A preferred embodiment of the present invention is an in vitro method for the delivery of antigens into the MHC class I presentation pathway, whereby the laboratory material is selected from the plastic materials used, including polystyrene or polyethylene, and in particular Eppendorf tubes, Falcon tubes, pipettes, test tubes, pipette tips, microtiter plates and petri dishes.

A preferred embodiment of the present invention is an in vitro method for the delivery of antigens into the MHC class I presentation pathway, whereby incubation temperature ranges from 0° C. to 37° C., mainly from 4° C. to 25° C. and in a preferred embodiment is room temperature (approximately 20° C.).

A preferred embodiment of the present invention is an in vitro method for the delivery of antigens into the MHC class I presentation pathway resulting in the activation of antigen-specific CD8⁺ T cells. Methods suitable for documenting the activation of antigen-specific CD8⁺ T cells include measurement of the production of cytokines such as IFN-γ, TNF-α and IL-2; proliferation tests; cytotoxicity tests, and other methods that are known to a person skilled in the art and are described in the literature.

A particularly preferred embodiment of the present invention is an in vitro method for the delivery of antigens into the MHC class I presentation pathway comprising the following steps: (a) any separation or concentration that is necessary for antigen-presenting cells using density gradient centrifugation isolation and/or cell sorting and/or magnetic bead isolation and/or adherence and/or other methods known to a person skilled in the art, and adjusted cell concentrations ranging from 10⁵ to 10⁹ cells/ml; (b) adding PI in concentrations ranging from 1 to 10 μg/ml to the antigen-presenting cells; (c) adding LLO in concentrations ranging from 1 to 5000 ng/ml, (d) incubation for 1-45 minutes at room temperature; (e) measurement of PI inflow into the cells during or after LLO incubation; (f) quantisation of specific parameters for which PI inflow into antigen-presenting cells can be measured; and (g) delivery of an antigen into the MHC class I presentation pathway of the antigen-presenting cells, including direct transfer of the specific parameters determined in step (f), and bringing the cells into contact with Listeriolysin (LLO) and the delivered antigens.

An additional embodiment of the in vitro method of the present invention allows for the determination of relevant parameters by measuring PI inflow into cells kept in PBS buffer during the experiment. The transfer of the relevant parameters from the preliminary experiment to the presentation experiment (whereby PBS is substituted for cell culture medium) leads to improved MHC class I antigen presentation in several cell types.

An additional embodiment of the present invention relates to a method for the generation of an APC that presents antigen epitopes from delivered exogenous antigens with MHC class I, including a method for the delivery of antigens to the MHC class I presentation pathway of antigen-presenting cells as described above, culminating in cultivation and/or isolation of MHC class I antigen-presenting APCs. Isolation can be realized using antibodies that are oriented toward defined peptide loaded MHC class I molecules (Porgador A., Yewdell J. W., Deng Y., Bennink J. R., and Germain R. N. 1997. Localisation, quantication, and in situ detection of specific peptide-MHC class I complexes using a monoclonal antibody. Immunity 6: 715), or other methods known to a person skilled in the art.

Another embodiment of the present invention relates to a method for the production of cytotoxic CD8⁺ T cells, including a method for delivery of antigens into the MHC class I presentation pathway of antigen-presenting cells as described above, and the isolation of cytotoxic CD8⁺ cells. Isolation of IFN-γ-secreting CTLs can be realized by using magnetic beads or FACS sorting (e.g. as described by Oelke et al 2001. Functional analysis of antigen-specific CTLs after enrichment based on cytokine secretion in: Miltenyi Biotec MACS & more newsletter Vol. 5, No. 1 2001; Oelke et al. 2000. Functional characterization of CD8(+) antigen-specific cytotoxic T lymphocytes after enrichment based on cytokine secretion: comparison with the MHC-tetramer technology. Scand. J. Immunol. 2000 December 52(6): 544-9), or another method known to a person skilled in the art and described in the literature.

Another embodiment of the present invention relates to a method for the characterisation of antigen epitopes presented by MHC class I molecules, including a method for the delivery of antigens into the MHC class I presentation pathway of antigen-presenting cells as described above, and isolation and characterisation of the presented antigen epitope as described by Purcell A. W. (Isolation and characterisation of naturally processed MHC-bound peptides from the surface of antigen-presenting cells. Methods Mol. Biol. 2004; 251: 291-306) und Torabi-Pour N. et al. (Comparative Study of Peptides Isolated from Class I Antigen Groove of Urological Specimens. A Further Step toward the Future of Peptide Therapy in Bladder Cancer Patients. Urologia Internationalis 2002; 68:183-188) (inter alia) or other methods known to a person skilled in the art.

Another embodiment of the present invention relates to a pharmaceutical composition comprising an APC as described above, a cytotoxic CD8⁺ T cell as described above, and/or a peptide comprising an MHC class I presented antigen epitope as described above, combined with pharmaceutically acceptable excipients. The active components according to the present invention can be processed using conventional buffers, solvents, tablet excipients, capsules, tablets, dropper solutions, suppositories, and injections and infusion preparations for purposes of peroral, rectal, or parenteral therapeutic applications.

Although the above method have been described as in vitro methods, the person of skill will be able to employ these methods in the context of a medical treatment involving either cytotoxic CD8⁺ T cells as described above or MHC class I presented antigen epitopes as described above. Another embodiment of the present invention relates to a method of treatment of cancerous diseases, comprising any of the above methods and providing the patient to be treated with an effective dose of the pharmaceutical composition according to the present invention. Cancers to be treated encompass all cancers that involve MHC class I antigen epitope presentation. Examples are described in the literature, such as, for example, Sundaram R, Dakappagari N K, Kaumaya P T. Synthetic peptides as cancer vaccines. Biopolymers. 2002; 66(3):200-16. Review. Moingeon P. Strategies for designing vaccines eliciting Th1 responses in humans. J Biotechnol. 2002 Sep. 25; 98(2-3):189-98. Ribas A, Butterfield L H, Glaspy J A, Economou J S. Cancer immunotherapy using gene-modified dendritic cells. Curr Gene Ther. 2002 February; 2(1):57-78. Apostolopoulos V, McKenzie I F, Wilson I A. Getting into the groove: unusual features of peptide binding to MHC class I molecules and implications in vaccine design. Front Biosci. 2001 Oct. 1; 6:D1311-20, and the references as cited therein.

A final embodiment of the present invention relates to a kit containing materials for the realization of an in vitro method for delivery of antigens into the MHC class I presentation pathway of cells as described above, for purposes of producing an APC that presents antigen peptide epitopes of extracellular proteins with MHC class I; for the production of cytotoxic CD8⁺ T cells as described above; and/or for the characterisation of MHC class I presented antigen epitopes as described above, whereby the method can be realized in a clinical setting. In addition to disposable materials, the kit materials comprise the relevant instruction manuals, which serve to ensure a high level of replicability for the method.

Hence, the present invention allows for rapid and sensitive cell-type specific determination of optimally efficacious LLO concentrations and incubation times for antigen delivery by measuring parameters such as the inflow of propidium iodide (PI) and other fluorescent compounds during or after LLO activity under defined conditions. By transferring the exact parameters that allow PI inflow to the LLO treatment process before or during antigen incubation, the present invention eliminates the parameter dependency that can have a persistently adverse impact on effective LLO concentration.

In the following, the invention is further elucidated using practical examples and Figures.

### DESCRIPTIONS OF THE FIGURES

FIG. 1: Mean PI fluorescence intensity of EL-4 cells with various LLO concentrations at various points in time
(a) Cell population in Dot Blot FSC (forward scatter) relative to PI
(b) Mean PI fluorescence intensity in relation to time

FIG. 2: MHC class I presentation of OVA following EL-4 cell incubation using various LLO concentrations

FIG. 3: MHC class I presentation of OVA following EL-4 cell incubation using various LLO concentrations

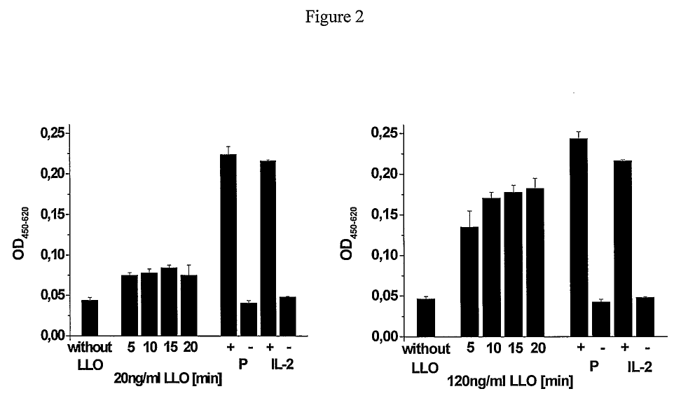

FIG. 4 Mean PI fluorescence intensity of EL-4 cells with various LLO concentrations at various points in time

FIG. 5 Mean PI fluorescence intensity of EL-4 cells with various LLO concentrations at various points in time
(a) Cell population in Dot Blot FSC relative to PI
(b) Mean PI fluorescence intensity in relation to time

FIG. 6: MHC class I presentation of OVA following LLO incubation of EL-4 cells based on published data

FIG. 7: Scattered light of monocytes and lymphocytes

Peripheral blood mononuclear cells (PBMCs) were classified as monocyte (R2) or lymphocyte (R1) populations on the basis of their scattered light parameters. PI inflow was measured separately for each population.

FIG. 8: PI inflow into monocytes (a) and lymphocytes (b) in the presence of LLO

FIG. 9: Percentage of IFN-γ secreting CD8⁺ T cells following pre-treatment with LLO and stimulation with CMVpp65

FIG. 10: PI inflow into monocytes (a) and lymphocytes (b) following LLO incubation

FIG. 11: Proportion of IFN-γ secreting CD8⁺ T cells following pre-treatment with LLO and stimulation with CMV lysate

FIG. 12: PI inflow into monocytes (a) and lymphocytes (b) in the presence of LLO

FIG. 13: Percentage of IFN-γ secreting CD8⁺ T cells following pre-treatment with LLO and stimulation with influenza antigen

### EXAMPLES

### Example 1

### Measurement of LLO-Mediated PI Inflow Prior to LLO Incubation for Antigen Delivery

In this experiment, EL-4 cell line was used as antigen-presenting cells (APCs). Tumor cell line EL-4 (H-2 Kb) was obtained from a C57BL/6 mouse via bezanthracene induced carcinogenesis (Ghose T., Guclu A., Tai J., Norvell S. T., and MacDonald A. S. 1976. Active immunoprophylaxis and immunotherapy in two mouse lymphoma models. J. Natl. Cancer Inst. 57(2): 303-15; Talmage D. W., Woolnough J. A., Hemmingsen H., Lopez L. and Lafferty K. J. 1977. Activation of cytotoxic T cells by nonstimulating tumor cells and spleen cell factor(s). Proc. Natl. Acad. Sci. USA. 74(10): 4610-4). However, it would be clear to a person skilled in the art that this method can be modified without any difficulty for use with other antigen-presenting cells. The Listeriolysin was produced as described by Darji et al (J. Biotechnol. 1995 Dec. 15; 43(3): 205-12).

### A. PI Inflow Measurement in EL-4 Cells During LLO Activity

In this experiment, PI inflow was measured for LLO activity with two different concentrations (20 ng/ml and 120 ng/ml). EL-4 cell concentration was adjusted to 2×10⁶/ml using RPMI/25 mM HEPES/5% FCS at room temperature. 20 μl PI (stock solution 100 μg/ml, end concentration 2 μg/ml), 409 μl RPMI/25 mM HEPES/5% FCS and 71 μl of LLO diluted in RPMI/25 mM HEPES/5% FCS (20 ng LLO) were added to 500 μl of the cell suspension (specimen volume 1 ml with 20 ng/ml LLO). In the same manner 20 μl PI, 52 μl RPMI/25 mM HEPES/5% FCS and 428 μl of LLO diluted in RPMI/25 mM HEPES/5% FCS (120 ng LLO) were added to 500 μl of the cell suspension. Addition of LLO constituted minute zero from which PI inflow was detected via flow cytometric analysis (BD FACScalibur). The measurement of mean PI fluorescence intensity was repeated at the times indicated.

The results of the experiment are shown in FIG. 1. The mean PI fluorescence intensity of EL-4 cells was determined following incubation at room temperature in RPMI/25 mM HEPES/5% FCS for 0, 5, 10, 15 and 20 minutes in the presence of 20 and 120 ng/ml LLO. No PI inflow into EL 4 cells was detected at any measuring point in the presence of 20 ng/ml LLO. After 5 minutes of incubation in the presence of 120 ng/ml LLO, incipient PI inflow was detected, and increased as LLO incubation continued.

B. Transfer of LLO Concentration and Incubation Time to Antigen Delivery into the MHC Class I Presentation Pathway

LLO mediated antigen delivery into the MHC class I presentation pathway was achieved under the same conditions that were used for measuring PI inflow in the preliminary experiment. EL-4 cell concentration was adjusted to 2×10⁶/ml using RPMI/25 mM HEPES/5% FCS at room temperature. As in the measurement of PI inflow, each batch of 1×10⁶ EL-4 cells was incubated using the same LLO volume and concentration and for the same LLO incubation times as specified in A (above), but without PI. Batch volume was adjusted by adding 20 μl RPMI/25 mM HEPES/5% FCS, and a non-LLO batch was also tested as a control. The LLO concentrations used for antigen delivery into the MHC 1 pathway were those for which in the preliminary experiment incipient PI inflow measured by increasing PI fluorescence intensity (120 ng/ml) was observed (Table 1).

TABLE 1

| | | LLO | | | | |
|---|---|---|---|---|---|---|
| | | in RPMI 5% | RPMI 5% | EL-4 2 × 10⁶/ml | | |
| | LLO | FCS | FCS | in RPMI 5% FCS | | |
| | concentration | 25 mM Hepes | 25 mM Hepes | 25 mM Hepes | Time | |
| Batch | [ng/ml] | [μl] | [μl] | [μl] | [min] | Temperature |
| 1 | 0 | 0 | 500 | 500 | 5 | RT |
| 2 | 120 | 428 | 72 | 500 | 5 | RT |
| 3 | 120 | 428 | 72 | 500 | 10 | RT |
| 4 | 120 | 428 | 72 | 500 | 15 | RT |
| 5 | 120 | 428 | 72 | 500 | 20 | RT |
| 6 | 0 | 0 | 500 | 500 | 5 | RT |
| 7 | 20 | 71 | 429 | 500 | 5 | RT |
| 8 | 20 | 71 | 429 | 500 | 10 | RT |
| 9 | 20 | 71 | 429 | 500 | 15 | RT |
| 10 | 20 | 71 | 429 | 500 | 20 | RT |

After stopping the reactions at the times indicated (by adding 3 ml RPMI/25 mM HEPES/5% FCS), each batch of cells was centrifuged and then re-suspended in 1 ml RPMI/25 mM HEPES/5% FCS. Following this, triplicates of each batch consisting of 100 μl/well (per 1×10⁵ EL-4 cells) were transferred to a 96-well plate cell and were cultivated for 20 hours using 500 μg/ml ovalbumin (OVA, 45 kDA) and 5×10⁴ B3Z cells/well.

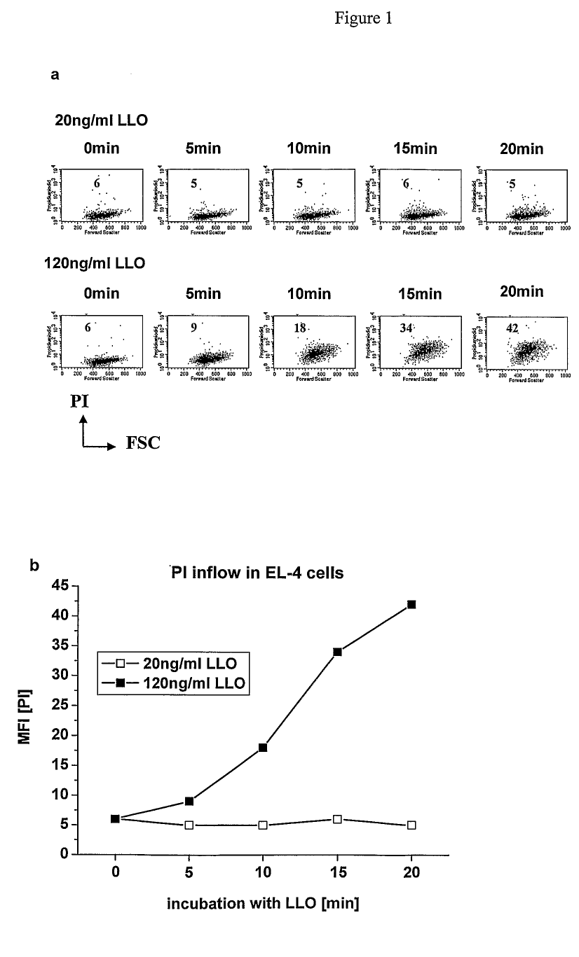

B3Z cells were used as reactive T cells in murine test systems (Karttunen J. and Shastri N. 1991. Measurement of ligand induced activation of single viable T-cells using the lacZ reporter gene. Proc. Natl. Acad. Sci. USA 88: 3972-76). This CD8⁺ T cell hybridoma line has a T cell receptor (TCR) that is specific for OVA peptide 257-264 as presented by MHC class I molecule H-2K^{b}. When B3Z cell TCRs interact with OVA peptide 257-264 presenting H-2K^{b}-molecules, they secrete IL-2. Untreated EL-4 cells with and without 1 μg/ml OVA 257-264 (P) and B3Z cells were incubated as controls. Following each of the incubation times indicated, 2×50 μl culture supernatant was removed from each well and transferred to a 96-well plate cell. IL-2 supernatant content was measured via proliferation of the IL-2 dependent T cell line CTLL-2. CTLL-2 cells with and without IL-2 were used as controls for this proliferation test. CTLL-2 cell proliferation was documented as an OD value using a Roche proliferation test (cell proliferation reagent cat. no. 1 644 807). The results of the test are shown in FIG. 2.

Hence in this example, no PI (A) inflow and only negligible MHC class I presentation of OVA (B) was observed following EL-4 cell incubation in 20 ng/ml LLO. On the other hand, steadily increasing PI (A) inflow and a high level of MHC class I presentation of OVA (B) was observed following EL-4 cell treatment using 120 ng/ml OVA. In this example, successful delivery of extracellular OVA to the MHC class I presentation pathway of EL-4 cells was realized concurrently with the measurement of detectable LLO mediated PI inflow into the cells.

### Example 2

### Measurement of LLO Dependent Mean PI Fluorescence Intensity Concomitantly with LLO Incubation for Antigen Delivery into the MHC Class I Presentation Pathway

EL-4 cells were used as APCs in this experiment. PI inflow was measured concomitantly with LLO incubation for the purpose of antigen delivery. EL-4 cell concentration was adjusted to a cell concentration of 2×10⁶/ml using RPMI/25 mM HEPES/5% FCS at room temperature. 1×10⁶ EL-4 cells were incubated for each batch using various LLO concentrations and incubation times (Table 2).

After stopping the reactions at the times indicated (by adding 3 ml RPMI/25 mM HEPES/5% FCS), the cells were centrifuged and then re-suspended in 1 ml RPMI/25 mM HEPES/5% FCS. Following this, triplicates of each batch consisting of 100 μl/well (per 1×10⁵ EL-4 cells) were transferred to a 96-well plate cell and were cultivated for 20 hours using 500 μg/ml ovalbumin (OVA, 45 kDA) and 5×10⁴ B3Z cells/well. MHC class I presentation of OVA (45 kDA) was documented using the method described in example 1. The results of the test are shown in the illustration.

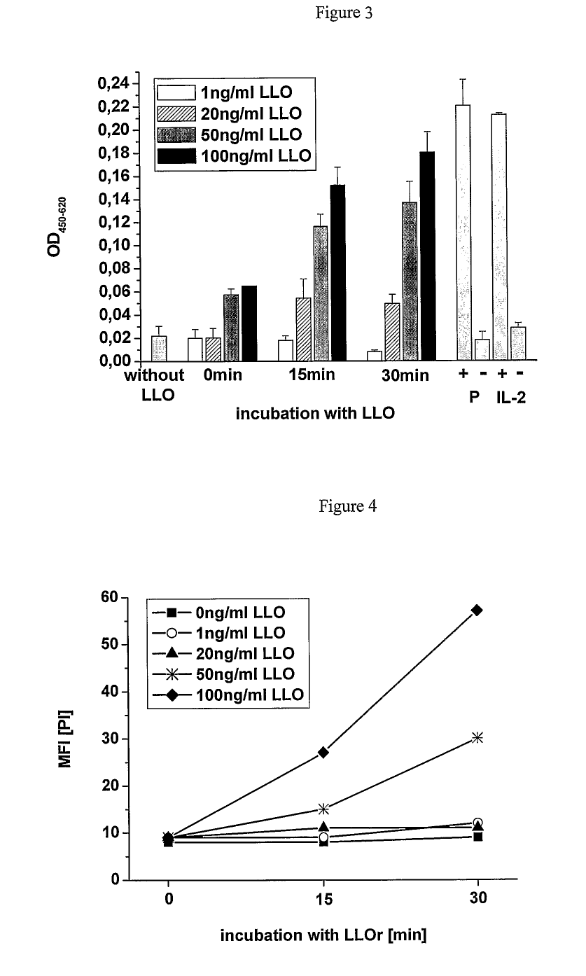

During cell incubation for purposes of OVA delivery into the MHC class I presentation pathway (Table 2), a duplicate of each batch was treated with PI and was measured at the times indicated in order to determine mean PI fluorescence intensity (Table 3). The results are shown in FIG. 4.

TABLE 2

| | | LLO in | RPMI 5% | EL-4 2 × 10⁶/ml | | |
|---|---|---|---|---|---|---|
| | LLO | RPMI 5% FCS | FCS 25 mM | RPMI 5% FCS | | |
| | concentration | 25 mM HEPES | HEPES | 25 mM HEPES | Time | |
| Batch | [ng/ml] | [μl] | [μl] | [μl] | [min] | Temperature |
| 1 | 0 | 0 | 500 | 500 | 0 | RT |
| 2 | 1 | 36 | 464 | 500 | 0 | RT |
| 3 | 20 | 71 | 429 | 500 | 0 | RT |
| 4 | 50 | 179 | 321 | 500 | 0 | RT |
| 5 | 100 | 360 | 140 | 500 | 0 | RT |
| 6 | 1 | 36 | 464 | 500 | 15 | RT |
| 7 | 20 | 71 | 429 | 500 | 15 | RT |
| 8 | 50 | 179 | 321 | 500 | 15 | RT |
| 9 | 100 | 360 | 140 | 500 | 15 | RT |
| 10 | 1 | 36 | 464 | 500 | 30 | RT |
| 11 | 20 | 71 | 429 | 500 | 30 | RT |
| 12 | 50 | 179 | 321 | 500 | 30 | RT |
| 13 | 100 | 360 | 140 | 500 | 30 | RT |

In this example, no higher PI inflow than in the control was detected following EL-4 cell incubation in 1 and 20 ng/ml LLO and (as in example 1) MHC class I presentation of OVA was negligible at incubation minutes 15 and 30. On the other hand, incipient PI inflow was observed after 15 minutes of incubation in 50 ng/ml LLO, and this inflow underwent a marked increase by minute 30. The higher increase of mean PI fluorescence intensity using 50 ng/ml versus 20 ng/ml was also reflected by more pronounced MHC I OVA presentation at every measuring point. Pronounced PI inflow was observed after 15 minutes of incubation in 100 ng/ml LLO and increased steadily until minute 30. At the same time, efficacious OVA delivery into the MHC class I presentation pathway was observed following EL-4 cell incubation for 15 and 30 minutes in 100 ng/ml LLO.

TABLE 3

| | | LLO in | | | EL-4 2 × 10⁶/ | | |
|---|---|---|---|---|---|---|---|
| | | RPMI 5% | | RPMI 5% | ml | | |
| | | FCS | PI stock | FCS | RPMI 5% | | |
| | LLO | 25 mM | solution | 25 mM | FCS 25 mM | | |
| | concentration | HEPES, | 100 μg/ml | HEPES | HEPES | Time | |
| Batch | [ng/ml] | [μl] | [μl] | [μl] | [μl] | [min] | Temperature |
| PI 1 | 0 | 0 | 20 | 480 | 500 | 0, 15, 30 | RT |
| PI 2 | 1 | 36) | 20 | 444 | 500 | 0, 15, 30 | RT |
| PI 3 | 20 | 71 | 20 | 409 | 500 | 0, 15, 30 | RT |
| PI 4 | 50 | 179 | 20 | 301 | 500 | 0, 15, 30 | RT |
| PI 5 | 100 | 360 | 20 | 120 | 500 | 0, 15, 30 | RT |

In this example, no PI inflow (20 ng) and only a very low level of antigen delivery were observed. LLO treatment characterized by pronounced PI inflow (50 ng) culminated in efficacious delivery and MHC I presentation of exogenous antigens that increased concomitantly with LLO concentration.

### Example 3

### LLO Incubation in Accordance with Published Data

Unlike examples 1 and 2, in this example LLO incubation was realized on the basis of published data (Darji A., Chakraborty T., Wehland J., Weiss S. 1995. Listeriolysin generates a route for the presentation of exogenous antigens by major histocompatibility complex class I. Eur. J. Immunol. 25(10):2967-71, und Darji A., Chakraborty T., Wehland J., Weiss S. 1997. TAP-dependent major histocompatibility complex class I presentation of soluble proteins using listeriolysin. Eur. J. Immunol. 27(6):1353-9).

EL-4 cells were used as APCs and in accordance with the published data were incubated with LLO for purposes of antigen delivery into the MHC class I presentation pathway. The EL-4 cells were adjusted to a cell concentration of 2×10⁶/ml using RPMI/25 mM HEPES (37° C. without serum) and were incubated at 37° C. for 15 minutes using 1 μg/ml LLO and 100 μg/ml OVA. Non-LLO batches were tested as controls (table 4).

TABLE 4

| | | LLO in | | RPMI | EL-4 2 × 10⁶/ml | | |
|---|---|---|---|---|---|---|---|
| | LLO | RPMI 25 mM | OVA | 25 mM | RPMI 25 mM | | |
| | concentration | HEPES | [10 mg/ml] | HEPES | HEPES | Time | Temperature |
| Batch | [μg/ml] | [μl] | [μl] | [μl] | [μl] | [min] | [° C.] |
| 1 | 0 | 0 | 10 μl | 490 | 500 | 15 | 37 |
| 2 | 0 | 0 | 10 μl | 490 | 500 | 60 | 37 |
| 5 | 1 | 357 | 10 μl | 133 | 500 | 15 | 37 |
| 6 | 1 | 357 | 10 μl | 133 | 500 | 60 | 37 |

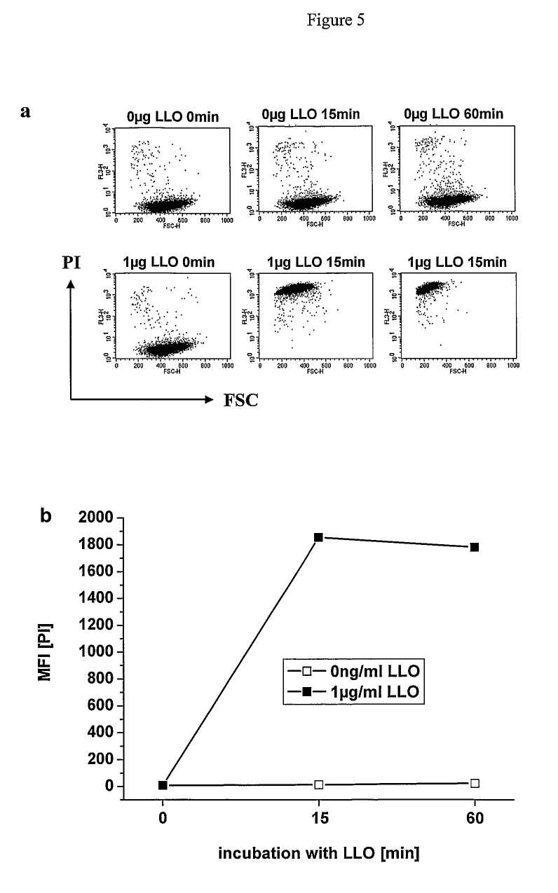

After stopping the reactions at the times indicated (by adding 3 ml RPMI/25 mM HEPES). the cells were centrifuged and then re-suspended in 1 ml RPMI/10% FCS. Following this, triplicates of each batch consisting of 100 μl/well (per 1×10⁵ EL-4 cells) were transferred to a 96-well plate cell and were cultivated for 20 hours with 5×10⁴ B3Z cells/well either in the presence of 500 μg/ml ovalbumin (OVA, 45 kDA) or without antigen. MHC class I presentation of OVA (45 kDA) was documented using the method described in example 1. The results of the cultivation of the LLO-treated cells with 500 μg/ml OVA were comparable to those obtained without cultivation in the presence of antigen (see FIG. 6). In both cases no antigen was presented by MHC I molecules.

During cell incubation for purposes of OVA delivery into the MHC class I presentation pathway (Table 4), a duplicate of each batch was measured at the times indicated in order to determine mean PI fluorescence intensity in FACScalibur. The results are shown in FIG. 5.

In the present example, no OVA delivery into the MHC class I presentation pathway was observed at minutes 15 or 60 of EL-4 cell incubation using LLO. Concurrently measured PI fluorescence intensity at minute 15 of serum-free cell incubation using 1 μg/ml LLO at 37° C. was substantially higher than PI inflow upon delivery into the MHC class I presentation pathway and did not increase further up to incubation minute 60 (FIG. 5). This extremely high PI inflow during LLO incubation in accordance with published data supports the view that the optimal LLO effective range was greatly exceeded in the previous examples and that LLO has a cytotoxic effect on the cells, thus forestalling further antigen processing or presentation.

### Example 4

### Example Using PBMCs

### Measurement of LLO-Mediated PI Inflow Prior to LLO Incubation for Antigen Delivery

In this example, human PBMCs were used as antigen-presenting cells. However, it would be clear to a person skilled in the art that this method can be modified without any difficulty for use with other antigen-presenting cells.

### Preparation

PBMCs were isolated from buffy coats originating from CMV seropositive donors using density gradient centrifugation. The various donor cells were either used immediately for testing or were divided into aliquots in autologous plasma and cryopreserved in 10% DMSO. In the following example, cryopreserved PBMCs were used for testing after being thawed.

A. Measurement of PI Inflow into PBMCs During LLO Activity:

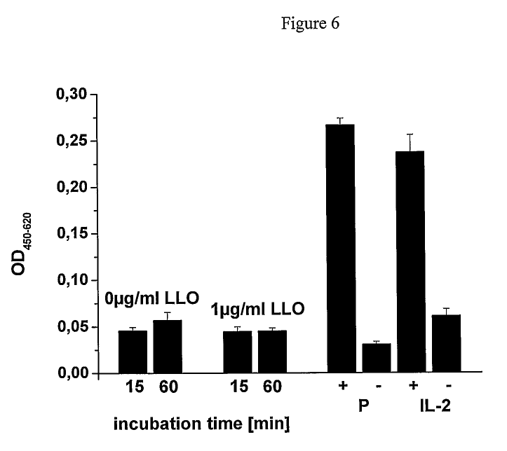

In this experiment, the PBMCs were adjusted to a 6×10⁶/ml cell concentration at room temperature using PBS/5% AB serum. 20 μl PI (stock solution 100 μg/ml, end concentration 2 μg/ml), 391 ml PBS/5% AB serum, and 89 μl LLO (25 ng LLO) diluted in PBS/% AB serum were pipetted into 500 μl of the cell suspension. Batch volume was 1 ml (25 ng LLO/ml) at the start of the experiment. Addition of LLO constituted minute zero from which PI inflow into the individual cell populations (monocytes and lymphocytes (see FIG. 7)) was detected via flow cytometric analysis.

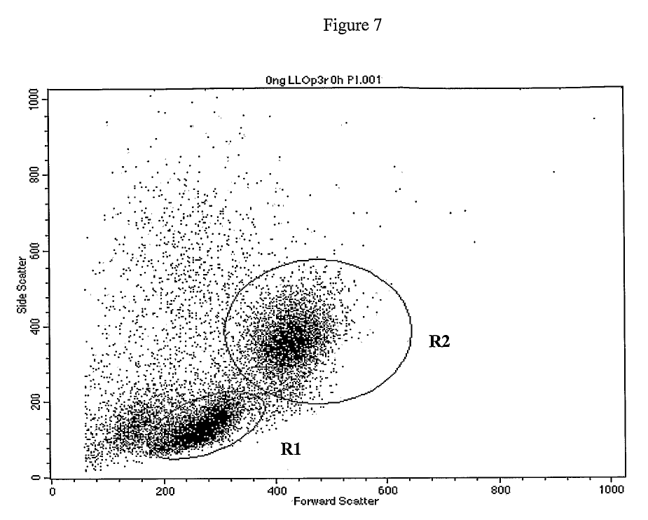

The subsequent measurements for PI inflow were realized at the times indicated. 25 ng/ml LLO-mediated PI inflow for monocytes and lymphocytes was measured at post-incubation minutes 2, 4, 6, 8, 10, 12, 14, 16 and 20 in PBS/5% AB serum at room temperature (RT). Increased PI inflow into the monocytes and lymphocytes was detected beginning from minute 10 following administration of LLO to the cells. The results are shown in FIG. 8.

### Comments on FIG. 8:

FIG. 8 shows LLO mediated PI inflow as a two histogram overlay, in each case at minute 0 with a subsequent measuring point. No PI is detected as long as the two histograms are fully covered. When the peak of each measuring point moves away from minute 0, PI inflow becomes increasingly detectable.

B. Transfer of LLO Parameters (LLO Concentration and Incubation Time) of Detectable PI Inflow to Antigen Delivery into the MHC Class I Presentation Pathway

LLO mediated antigen delivery into the MHC class I presentation pathway was achieved under the same conditions (apart from the buffer (PBS/5%/AB serum)) as for the prior measurement of PI inflow. The same cell concentrations, volumes, serum concentration and batches, plastic materials and LLO batches were also used. PI inflow was measured during LLO activity at the same temperature as the subsequent LLO incubation for exogenous antigen delivery.

TABLE 5

| | | LLO | | PBMCs | | |
|---|---|---|---|---|---|---|
| | LLO | in RPMI/5% AB | RPMI/5% | 10⁷/ml | | |
| | concentration | serum | AB serum | PBS | Time | |
| Batch | [ng/ml] | [μl] | [μl] | [μl] | [min] | Temperature |
| 1 | 0 | 0 | 500 | 500 | 0 | RT |
| 2 | 25 | 89 | 411 | 500 | 0 | RT |
| 3 | 25 | 89 | 411 | 500 | 4 | RT |
| 4 | 25 | 89 | 411 | 500 | 6 | RT |
| 5 | 25 | 89 | 411 | 500 | 8 | RT |
| 6 | 25 | 89 | 411 | 500 | 10 | RT |
| 7 | 25 | 89 | 411 | 500 | 12 | RT |
| 8 | 25 | 89 | 411 | 500 | 15 | RT |

The PBMCs under treatment were adjusted to a 6×10⁶/ml cell concentration using RPMI/5% AB serum. As in the preliminary tests, each 3×10⁶ batch of PBMCs was incubated in the same LLO volume and concentration, except without PI. The volume was replaced with RPMI/5% AB serum. A non-LLO batch was tested as a negative control. The LLO incubation times selected were those for which either no PI inflow into lymphocytes, the initiation of PI inflow into lymphocytes, or increasing inflow into lymphocytes could be observed during the preliminary test (FIG. 8 and Table 5).

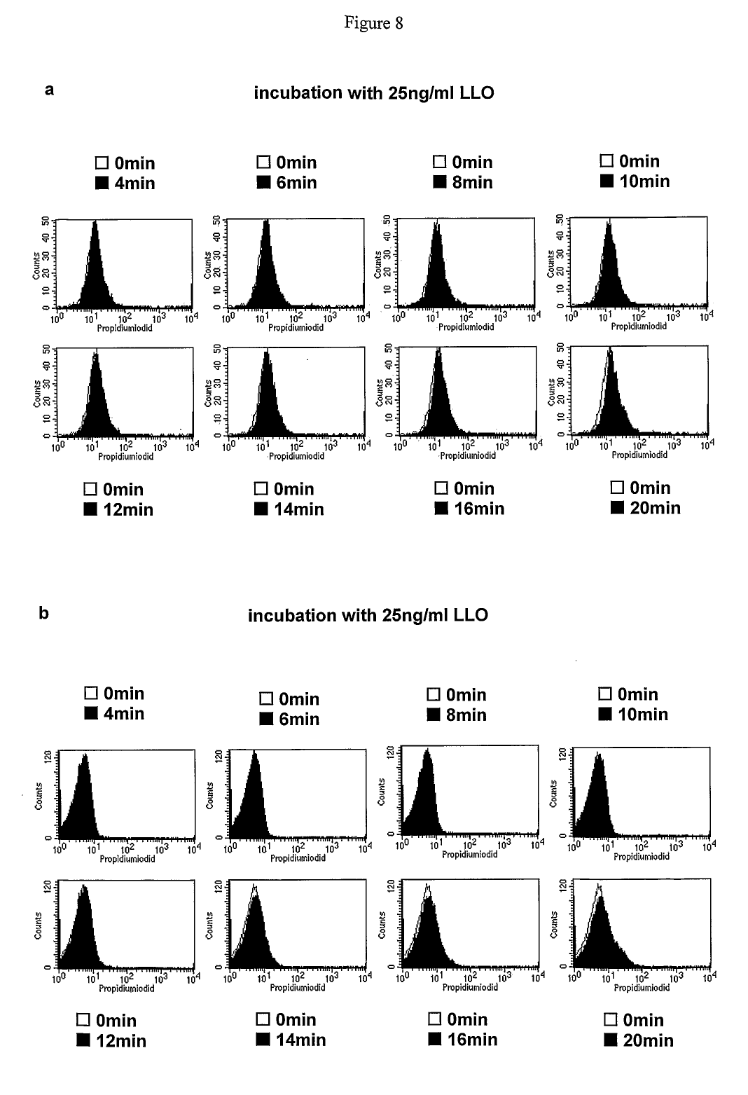

The reactions in the various batches were stopped at the times indicated by adding 3 ml RPMI/5% AB serum. The cells were centrifuged and then re-suspended in 280 μl RPMI/5% AB serum. Of the cell suspension, 150 μl/well (with 1.6×10⁶ cells) were transferred to a 96 well plate cell and cultivated for 16 hours in the presence of 2.5 μg/ml CMVpp65. After 16 h the cells were harvested and the percentage of CD8⁺ secreting T cells in the lymphocyte population was determined via flow cytometric analysis using IFN-γ secretion assays from Miltenyi Biotec (Germany) (FIG. 9).

Hence PI inflow was used to define the effective range where LLO can be utilized for antigen delivery (CMVpp65) into the cytosol including subsequent processing and MHC class I presentation.

In the example described here, an increased percentage of activated antigen specific CD8⁺ T cells was observed coterminously with the initiation of PI inflow into the cells (at minute 10 following the addition of LLO in the preliminary test using PBS/5% AB serum). As LLO incubation progressed, the percentage of antigen specific CD8⁺ T cells increased to its maximum level (at minute 10) relative to the control without LLO.

In incubations of longer duration, LLO displayed increasing cytotoxicity and the activation rate of antigen-specific CD8⁺ T cells decreased. Thus, the period of time within which LLO incubation allows for antigen delivery and processing can easily be determined by measuring LLO mediated PI inflow.

### Example 5

### Example Using PBMCs

### Measurement of PI Inflow Following LLO Activity

In this example, as in example 4, cryopreserved PBMCs from a CMV seropositive donor were thawed and used for testing. PI inflow was measured following LLO incubation of the PBMCs. The cells were adjusted to a 1×10⁷/ml cell concentration using RPMI/3% AB serum (RT). 5×10⁶ PBMCs were incubated in 30 ng/ml LLO per batch for each period indicated (Table 6).

TABLE 6

| | | LLO | RPMI/ | | | |
|---|---|---|---|---|---|---|
| | LLO | in RPMI/3% AB | 3% AB | PBMCs 10⁷/ml | | |
| | concentration | serum | serum | RPMI/3% AB serum | Time | |
| Batch | [ng/ml] | [μl] | [μl] | [μl] | [min] | Temperature |
| 1 | 30 | 107 | 393 | 500 | 0 | RT |
| 2 | 30 | 107 | 393 | 500 | 10 | RT |
| 3 | 30 | 107 | 393 | 500 | 15 | RT |
| 4 | 30 | 107 | 393 | 500 | 20 | RT |
| 5 | 30 | 107 | 393 | 500 | 25 | RT |
| 6 | 30 | 107 | 393 | 500 | 30 | RT |

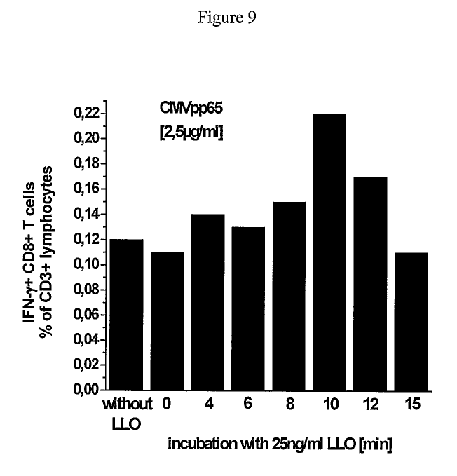

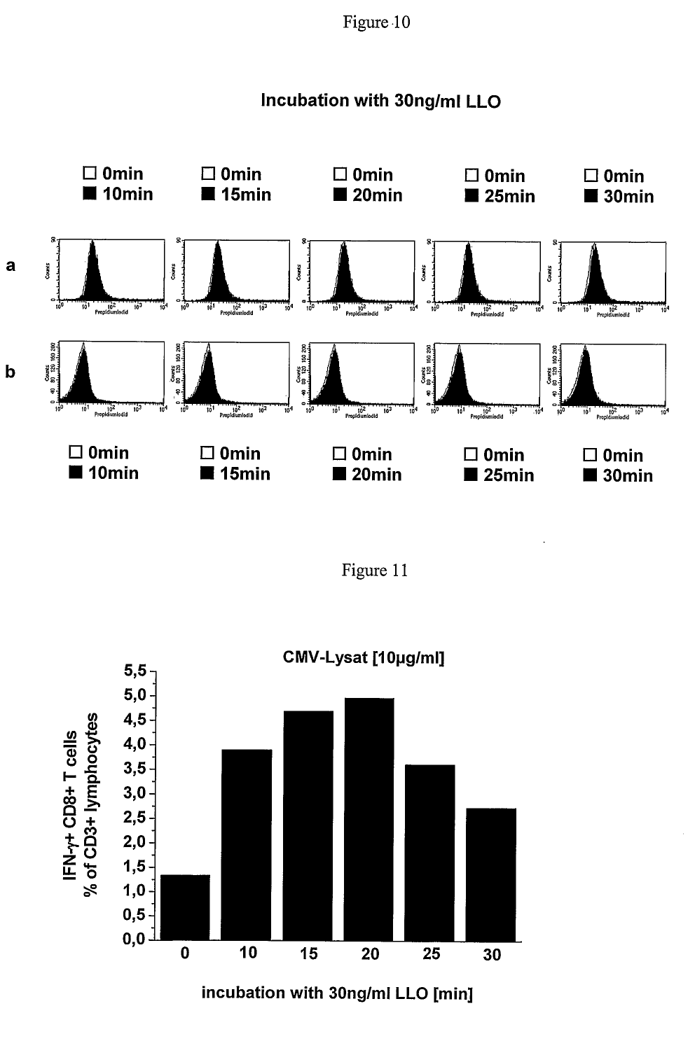

The reactions in the various batches were stopped at the times indicated by adding 3 ml RPMI/3% AB serum. 500 μl of the 4 ml per batch were transferred to another test tube after being carefully resuspended, and then underwent flow cytometric analysis after 2 μg/ml PI were added. The results are shown in FIG. 10 (see comments on FIG. 8, p. 31). The remaining cells were centrifuged and then re-suspended in 300 μl RPMI/3% AB serum. Following cell suspension, 150 μl/well (with 2.2×10⁶ cells) were transferred to a 96 well plate cell and cultivated for 16 hours using 10 μg/ml CMV lysate. The percentage of IFN-γ secreting CD8⁺ T cells was measured as described in example 4. The results are shown in FIG. 11.

In this example, PI inflow was detected at minute 10 following PBMC incubation in LLO, and this inflow increased as incubation progressed. Concurrently with PI inflow, an increase in the percentage of IFN-γ secreting CD8⁺ T cells was detected until LLO incubation minute 20. With longer LLO incubations, the proportion of detectable antigen specific CTLs decreased. This is attributable to the cytotoxic effect of LLO.

### Example 6

### Example Using PBMCs (Peripheral Blood Mononuclear Cells)

### Measurement of LLO-Mediated PI Inflow Prior to LLO Incubation for Antigen Delivery

In this example, PBMCs were used as antigen-presenting cells as was done in examples 4 and 5. The PBMCs were isolated from heparinized whole blood using density gradient centrifugation, and in contrast to examples 4 and 5, were immediately tested without prior cryogenation. Influenza antigens were used as stimulants for this experiment.

A. Measurement of PI Inflow into PBMCs During LLO Activity:

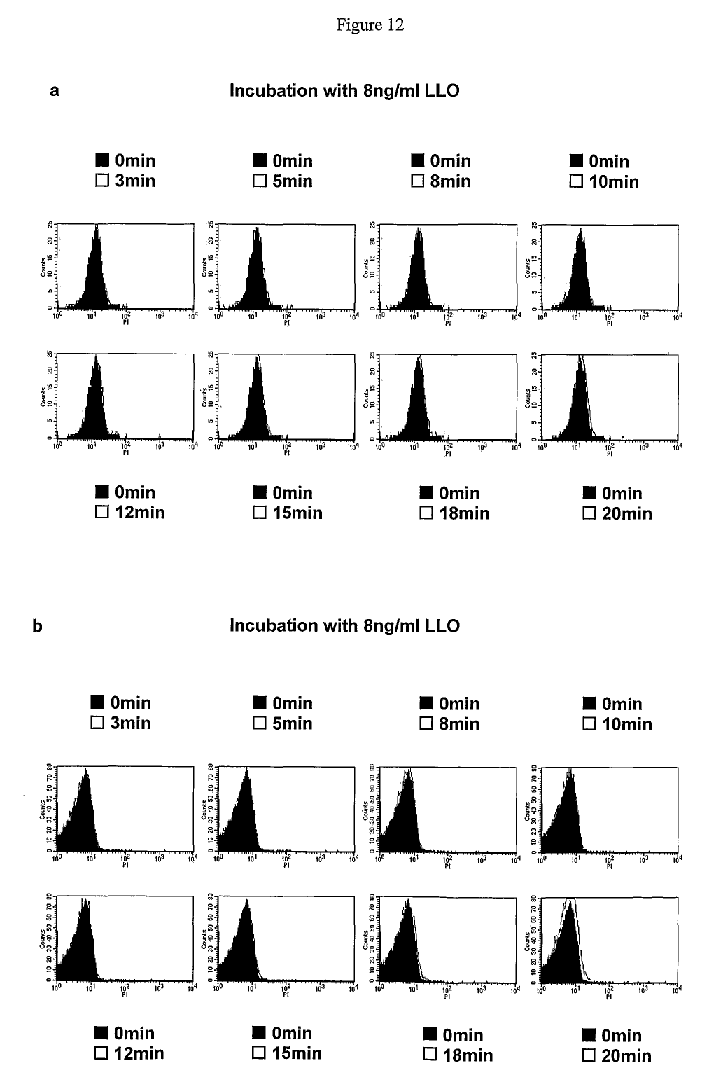

5×10⁵ PBMCs were incubated in PBS/5% AB serum at room temperature using 2 μg/ml PI and 8 ng LLO in 1 ml volume. In this process, 5×10⁵ PBMCs were resuspended in 694 μl PBS/5% AB serum. Following the addition of 20 μl PI (stock solution 100 μg/ml, end concentration 2 μg/ml) 286 μl LLO (diluted in PBS/5% AB serum) were added. Addition of LLO constituted minute zero from which PI inflow into the individual cell populations (monocytes and lymphocytes) was analyzed via flow cytometric analysis. The subsequent measurements for PI inflow were realized at the times indicated. Monocytes and lymphocytes incubated with 8 ng/ml LLO were measured at post-incubation minutes 3, 5, 8, 10, 12, 15, 18 and 20 in PBS/5% AB serum at room temperature (RT). Increased PI inflow into the monocytes and lymphocytes was detected beginning from minute 15 following administration of LLO to the cells. The results are shown in FIG. 12 (see comments on FIG. 8, p. 31).

B. Transfer of the Parameters (LLO Concentration and Incubation Time) for Detectable PI Inflow to Antigen Delivery into the MHC Class I Presentation Pathway

As in example 4, the parameters from the preliminary test were transferred to LLO incubation for antigen delivery. The PBMCs under treatment were adjusted to a 1×10⁶/ml cell concentration using RPMI/5% AB serum. As in the preliminary experiment, 5×10⁵ PBMCs per batch were incubated using mit 8 ng/ml LLO, and were also incubated without LLO. Measuring points selected were (a) when incipient PI inflow could be detected (minute 15); and (b) when PI inflow was clearly detectable (20 minutes) (Table 7, FIG. 12).

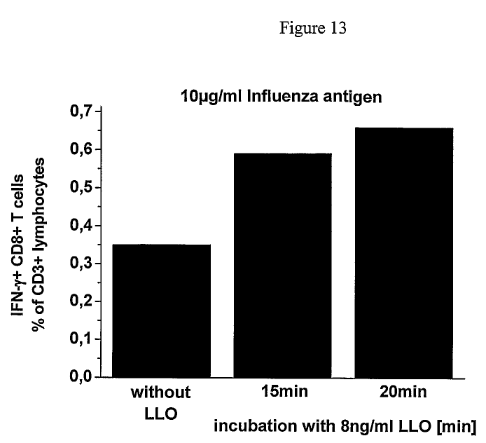

The reactions in the various batches were stopped at the times indicated by adding 3 ml RPMI/5% AB serum. Following centrifugation, the cells were resuspended in 100 μl RPMI/5% AB serum with 10 μg/ml influenza antigen (batches 1-3) and as a control, without antigens (batches 4 and 5) and were transferred to a microtiter plate. After being cultured for 16 hours, the cells were harvested and the percentage of IFN-γ secreting CD8⁺ T cells in the lymphocyte population was determined as described in example 4 (FIG. 13). Hence, when the parameters of the preliminary test (A) were transferred to the LLO-mediated delivery of antigen (B), an increase in the percentage of activated antigen specific CTLs was observed concurrently with incipient PI inflow (minute 15) and in the presence of demonstrable PI inflow (minute 20).

TABLE 7

| | | LLO | | PBMCs 10^{6/}ml | | |
|---|---|---|---|---|---|---|
| | LLO | in RPMI/5% AB | RPMI/5% AB | RPMI/5% AB | | |
| | concentration | serum | serum | serum | Time | |
| Batch | [ng/ml] | [μl] | [μl] | [μl] | [min] | Temperature |
| 1 | 0 | 0 | 500 | 500 | 15 | RT |
| 2 | 8 | 286 | 214 | 500 | 15 | RT |
| 3 | 8 | 286 | 214 | 500 | 20 | RT |
| 4 | 0 | 0 | 500 | 500 | 15 | RT |
| 5 | 8 | 286 | 214 | 500 | 15 | RT |

1. An in vitro method for the delivery of an exogenous antigen to the MHC class I presentation pathway, comprising the following steps:
a) Preparation of suitable antigen-presenting cells.
b) Determination of suitable specific parameters for the antigen-presenting cell method characterized by the following: (1) bringing the cells into contact with a haemolysin such as Listeriolysin (LLO) and a marker substance; (2) measurement of substance inflow into the cells; and (3) realizing any necessary adjustment of the specific parameters, and
c) Antigen delivery into the MHC class I presentation pathway of the cells, characterized by direct transfer of the parameters determined in step b), characterized by bringing the cells into contact with Listeriolysin (LLO) and the antigen under delivery.

2. The in vitro method for the delivery of an extracellular antigen to the MHC class I presentation pathway of cells according to claim 1 characterized in that the specific parameters for the method are selected from a group consisting of:
the cell lines or primary cells to which the antigen is to be delivered,
the quality of the cells used,
the cell concentration used,
batch volume,
the culture medium and/or buffer used,
the presence or absence of serum components during incubation,
serum and/or cholesterol concentration,
the antigen under delivery.
the laboratory material used during the experiments,
incubation temperature,
cell line or primary cell LLO incubation time,
LLO type,
LLO purification method,
LLO concentration used, and
LLO redox status.

3. The in vitro method for antigen delivery into the MHC class I presentation pathway of cells according to claim 1, characterized in that one additional specific parameter is constituted by antigen size, load and/or amino acid composition.

4. The in vitro method for antigen delivery into the MHC I presentation pathway according to claim 1, characterized in that the laboratory material used consists of plastic materials as well as Eppendorf tubes, Falcon tubes, pipettes, test tubes, pipette tips, microtiter plates and petri dishes.

5. The in vitro method for antigen delivery into the MHC class I presentation pathway of cells according to claim 1, characterized in that the cell line or primary cells into which the antigen is delivered is selected from primary human and murine cells; cell lines; peripheral blood mononuclear cells (PBMCs), monocytes or lymphocytes; immature monocyte derived DCs; the THP-2 cell line, mouse bone marrow in suspension; adherent mouse bone marrow macrophages; the EL-4 cell line; and the NIH3T3 cell line.

6. The in vitro method for antigen delivery into the MHC class I presentation pathway of cells according to claim 1, characterized in that the antigen is up to 550 kDa in size.

7. The in vitro method for antigen delivery into the MHC class I presentation pathway of cells according to claim 1, characterized in that the antigen is selected from proteins or parts thereof whose antigen epitope is to be presented with MHC I.

8. The in vitro method for antigen delivery into the MHC class I presentation pathway of cells according to claim 1, characterized in that cell concentration ranges from 10⁵/ml to 10⁹/ml.

9. The in vitro method for antigen delivery into the MHC class I presentation pathway of cells according to claim 1, characterized in that the LLO is derived from culture supernatant, purified LLO or recombinantly produced LLO.

10. The in vitro method for the antigen delivery into the MHC class I presentation pathway of cells according to claim 1, characterized in that LLO incubation time ranges from 1 minute to 16 hours.

11. The in vitro method for antigen delivery into the MHC class I presentation pathway of cells according to claim 1, characterized in that step b) is 30-45 minutes long.

12. The in vitro method for antigen delivery into the MHC class I presentation pathway of cells according to claim 1, characterized in that LLO concentration ranges from 1 to 500 ng/ml.

13. The in vitro method for antigen delivery into the MHC class I presentation pathway of cells according to claim 1, characterized in that the culture medium and/or buffer used is selected from PBS, DMEM and RPMI.

14. The in vitro method for antigen delivery into the MHC class I presentation pathway of cells according to claim 1, characterized in that incubation temperature ranges from 0 to 37° C.

15. The in vitro method for antigen delivery into the MHC class I presentation pathway of cells according to claim 1, characterized in that a dye and/or fluorescence compound is used as a marker.

16. The in vitro method for antigen delivery into the MHC class I presentation pathway of cells according to claim 1, characterized in that marker inflow is measured using fluorescence measurement.

17. The in vitro method for antigen delivery into the MHC class I presentation pathway of cells according to claim 1, characterized by the activation of antigen specific CD8⁺ T cells.

18. The in vitro method for antigen delivery into the MHC class I presentation pathway of cells according to claim 1, comprising the steps of
a) optionally, separation or concentration of antigen-presenting cells by means of density gradient centrifugation isolation and/or cell sorting, and adjusted cell concentrations ranging from 10⁵ to 10⁹ cells/ml;
b) incubation of antigen-presenting cells at room temperature in 1 to 10 μg/ml PI for periods ranging from 1 to 5 minutes;
c) adding LLO in concentrations ranging from 1 to 5000 ng/ml;
d) incubation for 1-45 minutes at room temperature;
3) determination of PI inflow into the cells using flow cytometric analysis;
f) determination of specific parameters that are suitable for the measurement of PI inflow for antigen-presenting cells;
c) subsequent antigen delivery into the MHC class I presentation pathway of the cells, characterized by direct transfer of the parameters determined in step b), characterized by bringing the cells into contact with listeriolysin (LLO) and the antigen under delivery.

19. A method for the production of an APC that presents antigen-derived peptides with MHC class 1, wherein said method comprises antigen delivery into the MHC class I presentation pathway of cell using the method according to claim 1, and isolation of MHC class I antigen-presenting APCs.

20. A method for the induction and/or activation and/or expansion of cytotoxic CD8⁺ T cell population, characterized by a method for wherein said method comprises antigen delivery into the MHC class I presentation pathway of cells using the method according to claim 1, and isolation of the activated cytotoxic CD8⁺ T cells.

21. A method for the measurement of MHC class I-presenting antigen epitopes, wherein said method comprises antigen delivery into the MHC class I presentation pathway of cells using the method according to claim 1, and isolation and characterization of the MHC class I-presenting peptides.

22. A pharmaceutical composition comprising:
i) an APC obtained by an in vitro method for the delivery of an exogenous antigen to the MHC class I presentation pathway, comprising the following steps:
a) Preparation of suitable antigen-presenting cells.
b) Determination of suitable specific parameters for the antigen-presenting cell method characterized by the following: (1) bringing the cells into contact with a haemolysin such as Listeriolysin (LLO) and a marker substance; (2) measurement of substance inflow into the cells; and (3) realizing any necessary adjustment of the specific parameters, and
c) Antigen delivery into the MHC class I presentation pathway of the cells, characterized by direct transfer of the parameters determined in step b), characterized by bringing the cells into contact with Listeriolysin (LLO) and the antigen under delivery,
ii) a cytotoxic CD8⁺ T cell according to claim 20 obtained by a method for the induction and/or activation and/or expansion of cytotoxic CD8⁺ T cell population, wherein said method comprises antigen delivery into the MHC class I using a method according to claim 1, and isolation of the activated cytotoxic CD8⁺ T cells and/or
iii) a peptide characterized by an MHC class I-presenting antigen epitope a method for the measurement of MHC class I-presenting antigen epitopes, wherein said method comprises antigen delivery into the MHC class I presentation pathway of cells using a method according to claim 1, and isolation and characterization of the MHC class I-presenting peptides,
in conjunction with pharmaceutically compatible excipients.

23. A kit containing materials for the realization of an in vitro method for antigen delivery into the MHC class I presentation pathway of cells using a method according to claim 1, for purposes of producing an APC with an MHC class I-presenting antigen epitope, for purposes of producing cytotoxic CD8⁺ T cells according to claim 20 and/or for measurement of MHC class I-presenting antigen epitopes, characterized in that the method is realizable in a clinical setting.
